(19) United States
(12) Patent Application Publication
(10) Pub. No.: US 2019/0343779 A1
(43) Pub. Date: Nov. 14, 2019

(54) Emodin for Use in Gastrointestinal Cancer Therapy

(71) Applicant: UNIVERSITY OF SOUTH CAROLINA, COLUMBIA, SC (US)

(72) Inventors: DAPING FAN, COLUMBIA, SC (US); ELIZABETH MURPHY, COLUMBIA, SC (US)

(21) Appl. No.: 16/398,471

(22) Filed: Apr. 30, 2019

Related U.S. Application Data
(60) Provisional application No. 62/668,835, filed on May 9, 2018.

(51) Int. Cl. A61K 31/122; A61K 31/513; A61K 31/519; A61K 31/506; A61K 38/00; A61K 9/00; A61P 35/00; A61P 29/00; C07K 16/30

(52) U.S. Cl. CPC A61K 31/122; A61K 31/513; A61K 31/519; A61K 31/506; C07K 16/3046; A61K 9/0053; A61K 9/0019; A61P 35/00; A61P 29/00; A61K 38/005

(57) ABSTRACT

Methods are described for use in treatment of gastrointestinal cancers such as colorectal cancers, gastric cancer, intestinal cancers, and adenocarcinomas. Methods utilize emodin in prevention or inhibition of tumorigenesis. Methods can encompass utilization of emodin as a therapy for gastrointestinal cancer as the sole treatment or in conjunction with other drugs or therapies useful in treating the pathology. Emodin is also useful in decreasing side effects of chemotherapy. Emodin may be utilized in a therapy protocol for gastrointestinal cancer therapy in combination with other chemotherapies such as 5 fluorouracil for mitigating side effects including inflammation and improving quality of life for chemotherapy patients.

### CROSS REFERENCE TO RELATED APPLICATION

This application claims filing benefit of U.S. Provisional Patent Application Ser. No. 62/668,835, having a filing date of May 9, 2018, entitled “Emodin for use in Colorectal Cancer Therapy,” which is incorporated herein by reference for all purposes.

### BACKGROUND

Gastrointestinal cancers include cancers throughout the gastrointestinal tract as well as accessory organs and are responsible for more cancer deaths than cancers of any other bodily system. For instance, colorectal cancer is one of the most common malignancies, accounting for approximately 1.36 million new cases worldwide annually. Intestinal tumors, including adenocarcinoma, sarcoma, carcinoid tumors, gastrointestinal stromal tumor, and lymphoma, can go unnoticed for long periods of time and as such are often not discovered until late-stage.

5 fluorouracil (5FU) has been the first-choice chemotherapy drug for colorectal cancer for many years. Unfortunately, its clinical utility remains hindered by acquired resistance and toxicities resulting from its non-selectivity and consequential 10-15% success rate. Moreover, treatment side effects including fatigue, loss of appetite, and diarrhea, among others, often lead to discontinued or de-escalated use. While combination therapy has been employed to attempt to increase efficacy and reduce toxicity, they have had limited success. There is a need for identifying new strategies to increase efficacy and reduce toxicity of 5FU to prevent discontinuation or de-escalation of treatment.

There has been growing interest in herb-derived compounds in treatment protocols for gastrointestinal cancers as they can modulate multiple inflammatory and tumorigenic pathways, are inexpensive, and have low toxicity for chronic treatment. Emodin is a natural trihydroxy-anthraquinone which is found in several Chinese herbs including rhubarb (Rheum palmatum) and tuber fleece flower (Polygonum multiflorum, also commonly known as Chinese knotweed or he shou wu). Emodin has shown potential to inhibit inflammation in various settings. For instance, emodin has been shown to attenuate the severity of experimental disease models including arthritis, liver damage, atherosclerosis, myocardial ischemia, and breast cancer. For instance, it has been reported that emodin can reduce breast tumor growth and metastasis in mouse models.

What are needed in the art are gastrointestinal cancer therapies that can reduce tumorigenesis. What also are needed in the art are gastrointestinal cancer therapies that can decrease undesirable side effects of existing chemotherapies such as 5FU.

### SUMMARY

According to one embodiment, disclosed is a method to inhibit growth of gastrointestinal tumors (e.g., colorectal tumors, intestinal tumors, adenocarcinomas) that includes delivering emodin to an area that includes the gastrointestinal cancer cells. Delivery can be by any means and according to any methodology, e.g., systemically or locally.

According to another embodiment, disclosed is a method for treatment or prevention of side-effects of gastrointestinal cancer chemotherapy treatment. The method can include delivering emodin to a subject in conjunction with a second agent having efficacy for colorectal cancer chemotherapy, e.g., 5FU. The emodin can be delivered to the subject prior to, during, and/or following delivery of the second agent to the subject.

Also disclosed is a method for downregulating expression of proteins that are involved in inflammation and that are upregulated by treatment with a second agent. The method can include delivering emodin to a subject diagnosed with a gastrointestinal cancer. The method also can include delivering a second agent having efficacy for cancer chemotherapy to the subject, this second agent upregulating expression of one or more proteins that are involved in inflammation, e.g., MCP-1, TNF-α, and/or NOS2. The emodin can be delivered to the subject prior to, during, and/or following delivery of the second agent and, following the delivery, the expression of the one or more inflammatory proteins can be downregulated.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates the inhibition of colon tumorigenesis in a mouse model. Mice were administered emodin (40 mg/kg) during azoxymethane (AOM)/dextran sodium sulfate (DSS) treatment. Total polyp number was determined (n=12). *p<0.05.

FIG. 2 illustrates the inhibition of intestinal tumorigenesis in an Apc^{min/+} mouse model. Mice were administered dietary emodin or a vehicle treatment (n=7/group) via gavage at a dose of 40 mg/Kg three days a week for 12 weeks (*P<0.05).

FIG. 3 illustrates the reduction of tumor weight in an MC38 model. Mice were administered dietary emodin or vehicle treatment (n-4/group) via gavage at a dose of 40 mg/Kg three days a week for 4 weeks (#P<0.1).

FIG. 4 illustrates the inhibition of macrophage (MΦ) infiltration into tumors. MΦ infiltration into MC38 tumors (of similar size) was examined following control or emodin (40 mg/Kg) treatment using flow cytometry in an orthotopic model. (P<0.05; n=4).

FIG. 5 illustrates the inhibition of pro-tumor markers in a tumor microenvironment. MC38 tumors were assessed for pro-tumor markers. (*P<0.05; #P<0.1; n=4/group)

FIG. 6 illustrates the inhibition of 5FU-induced colon inflammation. 5FU treated mice were administered emodin (40 mg/kg 3×wk) or vehicle treatment (n=5). A non-disease phosphate buffer saline (PBS) group was used as a control (n=5). Following 5FU treatment inflammatory mediators were analyzed in the colon via q PCR. *p<0.05.

FIG. 7A illustrates the variation in mouse body weight following 3 cycles of 5FU treatment in combination with emodin (40 mg/kg 3×wk; n=9) or vehicle treatment (n=10). *p<0.05.

FIG. 7B illustrates pain sensitivity following 3 cycles of 5FU treatment with emodin (40 mg/kg 3×wk; n=9) or vehicle treatment (n=10). *p<0.05.

FIG. 7C illustrates survival percentage following 3 cycles of 5FU treatment with emodin (40 mg/kg 3×wk; n=9) or vehicle treatment (n=10). *p<0.05.

FIG. 7D illustrates fatigue levels following 3 cycles of 5FU treatment with emodin (40 mg/kg 3×wk; n=9) or vehicle treatment (n=10). *p<0.05.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, disclosed herein are methods and materials that can be utilized in treatment of gastrointestinal cancers including, without limitation, gastric cancer, colorectal cancers, adenocarcinomas, intestinal cancers of the large or small intestine, anal cancers, etc. More specifically, disclosed are methods for utilizing emodin in prevention or inhibition of gastrointestinal tumorigenesis as well as in decreasing side effects of cancer chemotherapy. As such, methods can encompass utilization of emodin in one embodiment as a therapy for a gastrointestinal cancer, e.g., colorectal cancer. Emodin may be administered as the sole treatment or in conjunction with other drugs or therapies useful in treating the pathology. For instance, emodin may be utilized in a therapy protocol for colorectal cancer therapy in combination with other chemotherapies including immunotherapies or cancer cell targeting therapies as are known in the art. Additional therapeutically effective agents can be administered as a component of a composition that includes emodin or in a separate composition, as desired.

Beneficially, it has been discovered that emodin can be utilized in mitigating side effects of other cancer chemotherapy agents, e.g., more traditional cancer chemotherapy agents. In one particular embodiment, emodin can be utilized in conjunction with the colorectal cancer chemotherapy agent 5FU. Other agents or combinations of agents for use with emodin include but are not limited to Avastin® (bevacizumab), Camptosar® (irinotecan, e.g., irinotecan hydrochloride), Xeloda® (capecitabine), Erbitux® (cetuximab), Cyramza® (ramucirumab), Eloxatin® (oxaliplatin), Wellcovorin® (leucovorin, e.g., leucovorin calcium), Lonsurf® (trifluridine and tipiracil hydrochloride), Opdivo® (nivolumab), Vectibix® (panitumumab), Stivarga® (regorafenib), Zaltrap® (Ziv-Aflibercept), or any combination thereof including, without limitation, CAPDX (capecitabine plus oxaliplatin), FOLFOX (5 fluorouracil, leucovorin, and oxaliplatin), FOLFIRI (leucovorin calcium, 5-fluorouracil, and irinotecan), FOLFIRI-bevacizumab, FOLFIRI-cetuximab, XELOX (oxaliplatin and capecitabine) FU-LV (fluorouracil and leucovorin calcium), XELIRI (capecitabine and irinotecan), etc.

Beneficially, emodin can inhibit inflammation that is a side-effect of other cancer chemotherapies. For example, colon inflammation is a common side effect of 5FU treatment that can lead to symptoms including diarrhea and abdominal pain. Addition of emodin to a treatment protocol, e.g., addition of dietary emodin following 5FU administration to a subject, can decrease symptoms of colon inflammation as compared to subjects that do not receive emodin in conjunction with the 5FU treatment.

Emodin can likewise inhibit functional deficits and mortality associated with cancer chemotherapy such as 5FU. For instance, combination of emodin with more traditional cancer chemotherapies such as 5FU can mitigate many chemotherapy side effects such as, and without limitation to, weight loss and pain sensitivity. As such, combination of emodin with a more traditional chemotherapy can decrease mortality of subjects undergoing treatment for colorectal cancers.

Without wishing to be bound to any particular theory, it is believed that beneficial aspects of emodin therapy in gastrointestinal cancer can be obtained through decreased expression of proteins that are involved in inflammation. In particular, utilization of emodin can lead to decreased expression of proteins that are involved in inflammation and that are upregulated in treatment with cancer chemotherapies. For example, treatment with emodin can lead to decreased expression of monocyte chemoattractant protein-1 (MCP-1/CCL2), which is a key chemokine that regulates migration and infiltration of monocytes/macrophages. MCP-1 has been demonstrated to recruit monocytes into foci of active inflammation (Ajuebor, et al., 1998), and has been shown to play a role in many cancers (Conti and Rollins, 2004), as well as other diseases including atherosclerosis (Namiki, et al., 2002), inflammatory bowel disease (Spoettl, et al., 2006), allergic asthma (lp, et al., 2006), rheumatoid arthritis (Rantapaa-Dahlqvist, et al., 2007), and diabetes (Kamei, et al., 2006).

Treatment with emodin also can lead to decreased expression of the cytokine tumor necrosis factor (TNF-α). TNF-α is a cytokine involved in systemic inflammation and is one of the positive acute phase proteins that make up the acute phase reaction in response to inflammation such as is caused by treatment with known chemotherapy agents such as 5FU.

Treatment with emodin also can lead to decreased expression of the enzyme nitric oxide synthase (NOS2). NOS2 is the principal enzyme involved in synthesis of nitric oxide (NO) and produces high level sustained NO synthesis by many cell types involved in inflammation (Coleman, 2001).

Accordingly, through combined treatment of gastrointestinal cancer with emodin and one or more other chemotherapy agents, e.g., 5FU, increased expression levels of inflammation related proteins can be mitigated, thereby decreasing inflammation related side-effects of chemotherapy agents.

In addition, treatment with emodin can lead to a decrease in macrophage infiltration into tumors. Macrophages have emerged as major players in the connection between inflammation and cancer. Tumor associated macrophages (TAMs) in the tumor microenvironment (TME), while mainly adopting a M2-like phenotype, play a direct role in promoting tumor progression. TAM represent up to 50% of the tumor mass and produce a wide array of inflammatory mediators with pro-tumoral functions. TAMs have been associated with colorectal cancer progression, metastasis, and poor prognosis. MΦ depletion can be effective at reducing colorectal tumor growth. Accordingly, a decrease in macrophage infiltration into tumors as can be brought about through emodin treatment can be of great benefit in cancer treatment.

Disclosed methods can be utilized in vivo for treatment of gastrointestinal cancer or in vitro for study of gastrointestinal cancer cells or tissue. In order for emodin to be effectively utilized in a clinical therapy, it can be delivered so as to be provided with suitable bioavailability. According to an in vivo treatment method, a composition including emodin and a pharmaceutically compatible carrier can be delivered to a subject via any pharmaceutically acceptable delivery system. In general, emodin may be administered to a subject according to known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Osmotic mini-pumps also may be used to provide controlled delivery of emodin through cannulae to the site of interest either systemically or locally, such as directly into a metastatic growth. In certain embodiments, emodin can be administered directly to the area of a tumor or cancer tissue, including administration directly to the tumor stroma during invasive procedures. Emodin also may be placed on a solid support such as a sponge or gauze for administration.

Pharmaceutically acceptable carriers include, but are not limited to, saline, buffered saline, glucose in saline, etc. Solid supports, liposomes, nanoparticles, microparticles, nanospheres or microspheres also may be used as carriers for administration of emodin. As used herein the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents also can be incorporated into the compositions.

The appropriate dosage (“therapeutically effective amount”) of the emodin can depend, for example, on the severity and course of the cancer, whether the emodin is administered for therapeutic purposes or in prevention of side effects of other chemotherapy agents, previous therapy, the patient's clinical history and response to the emodin, and the discretion of the attending physician, among other factors. Emodin can be administered to a subject at one time or over a series of treatments and may be administered to the subject at any time.

In one embodiment, a therapeutically effective amount of emodin can be in the range of about 0.5 mg/kg body weight/day to about 100 mg/kg body weight/day whether by one or more administrations, for instance in an animal subject, e.g., in a testing protocol. For example, emodin can be administered in an amount of from about 0.5 mg/kg body weight per day to about 50 mg/kg body weight/day, in some embodiments. For instance, a dosage amount for a human patient can be about 0.05-10 mg/kg/day in one embodiment. As expected, the dosage will be dependent on the condition, size, age and condition of the patient.

Emodin may be administered, as appropriate or indicated, in a single dose as a bolus or by continuous infusion, or as multiple doses by bolus or by continuous infusion. Multiple doses may be administered, for example, multiple times per day, once daily, multiple times per week, every 2, 3, 4, 5, 6 or 7 days, weekly, every 2, 3, 4, 5 or 6 weeks or monthly. However, other dosage regimens may be useful. The progress of this therapy is easily monitored by conventional techniques.

It can be advantageous to formulate oral or parenteral compositions in dosage unit form for ease of administration and uniformity of dosage. Dosage unit form as used herein includes physically discrete units suited as unitary dosages for the subject to be treated; each unit may contain a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

Pharmaceutical compositions for parenteral, intradermal, or subcutaneous injection can include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. A composition can contain minor amounts of auxiliary substances such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions and by the use of surfactants. A composition also may contain adjuvants such as preservatives, wetting agents, emulsifying agents and dispersing agents. It also may be desirable to include isotonic agents such as sugars, sodium chloride and the like.

For intravenous administration, suitable carriers include, without limitation, physiological saline, bacteriostatic water, Cremophor EL™ (BASF, Parsippany, N.J.), or phosphate buffered saline (PBS). In all cases, an injectable composition should be sterile and should be fluid to the extent that easy syringability exists. It must be stable under the conditions of manufacture and storage and must be preserved against the contaminating action of microorganisms such as bacteria and fungi. Prevention of the action of microorganisms may be ensured by the inclusion of various antibacterial and antifungal agents such as paraben, chlorobutanol, phenol, sorbic acid and the like. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions also can be prepared using a fluid carrier for use as a mouthwash, wherein the compound in the fluid carrier is applied orally and swished and expectorated or swallowed.

Pharmaceutically compatible binding agents and/or adjuvant materials can be included as part of an orally ingestible composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

When administered orally in liquid form, a liquid carrier such as water; petroleum; oils of animal or plant origin such as peanut oil, mineral oil, soybean oil, or sesame oil; or synthetic oils may be added. A liquid form may further contain physiological saline solution, dextrose or other saccharide solution, or glycols such as ethylene glycol, propylene glycol, or polyethylene glycol. When administered in liquid form, a composition can contain from about 0.5 to 90% by weight emodin, in one embodiment from about 1 to 50% by weight emodin.

For administration by inhalation, the emodin can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, a pharmaceutical composition can be formulated for sustained or controlled release of emodin. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially. Liposomal suspensions can also be used as pharmaceutically acceptable carriers. These can be prepared according to methods known to those skilled in the art.

It is to be understood that the in vivo methods have application for both human and veterinary use. The methods of the present invention contemplate single, as well as multiple administrations, given either simultaneously or over an extended period of time.

The present disclosure may be better understood with reference to the Examples set forth below.

### EXAMPLE 1

Effects of dietary emodin on tumorigenesis were examined in an azoxymethane (AOM)/dextran sodium sulfate (DSS) mouse model of colorectal cancer. Colorectal cancer was developed in mice according to known methods. Briefly, mice received a single injection of AOM (10 mg/Kg) followed by 2 cycles of DSS (2% in the water for 1 week followed by 2 weeks of plain water) as previously described (see, e.g., Tanaka T. Colorectal carcinogenesis: Review of human and experimental animal studies. J Carcinog 2009; 8:5; Enos R T, Velazquez K T, McClellan J L, et al. High-fat diets rich in saturated fat protect against azoxymethane/dextran sulfate sodium-induced colon cancer. American journal of physiology Gastrointestinal and liver physiology 2016; 310:G906-19).

The mice were administered either emodin or vehicle treatment (n=12) via gavage at a dose of 40 mg/Kg three days a week for the duration of the AOM/DSS protocol (10 weeks).

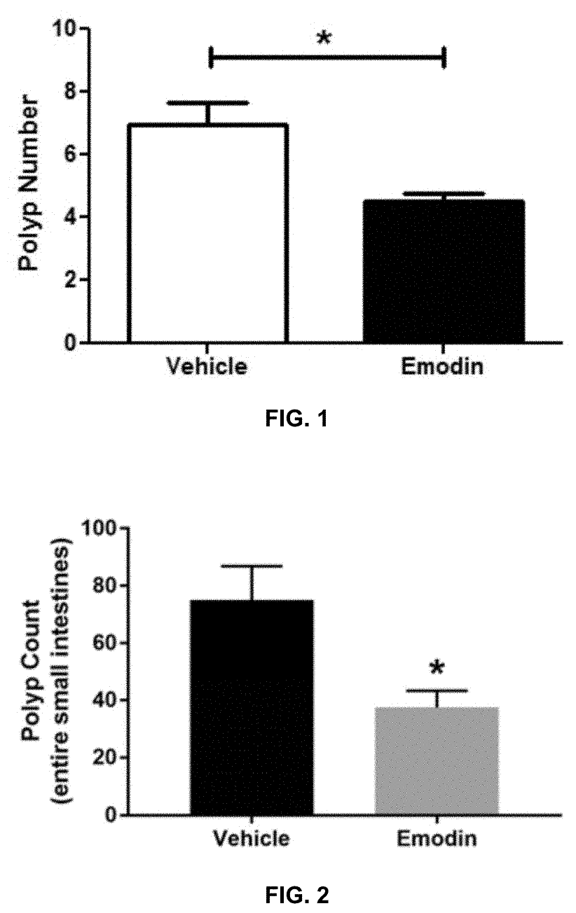

Results are illustrated in FIG. 1. As shown, the data indicate that emodin can significantly reduce tumorigenesis in mice. As shown, vehicle treated mice had approximately 7 tumors following the 10-week protocol, whereas emodin treated mice had approximately 5 tumors.

### EXAMPLE 2

The Apc^{min/+} model of intestinal tumorigenesis was utilized to test anti-tumor effects of emodin. Mice were administered emodin or vehicle treatment (n=7/group) via gavage at a dose of 40 mg/Kg three days a week for 12 weeks. Results are illustrated in FIG. 2. As indicated, the vehicle treated group had approximately 75 polyps per mouse, whereas emodin treated mice had less than 40 polyps per mouse.

### EXAMPLE 3

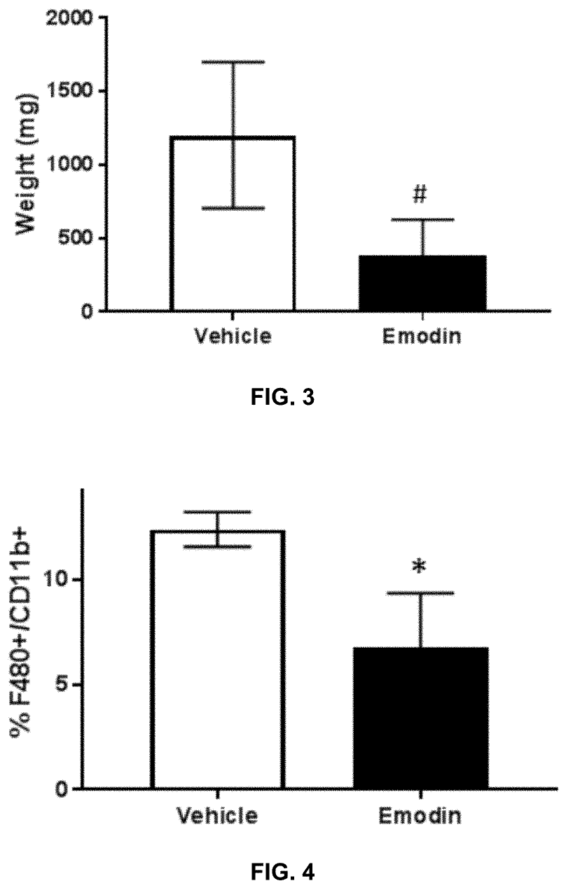

The MC38 mouse model of adenocarcinoma was used. Mice (n=4/group) were administered emodin or vehicle treatment via gavage at a dose of 40 mg/Kg three days a week for 4 weeks. Following the 4-week treatment period tumors in the vehicle treated group weighed approximately 1,200 mg, whereas those in the emodin treated group weighed approximately 400 mg. Results are shown in FIG. 3.

Taken together, Examples 1-3 indicate that emodin can significantly reduce tumorigenesis in all mouse models of tumorigenesis tested, including colorectal cancer, intestinal cancer, and adenocarcinoma.

### EXAMPLE 4

To test the effects of emodin on TAMs in colorectal cancer, macrophage infiltration into MC38 colon tumors following control or emodin (40 mg/Kg) treatment (3 times a week for 4 weeks) was examined using flow cytometry. Tumors of similar sizes were selected so that emodin effects could be determined independent of any changes in tumor size. As illustrated in FIG. 4, the data indicate that emodin can significantly reduce F4/80+ cells (macrophages) in colon tumors.

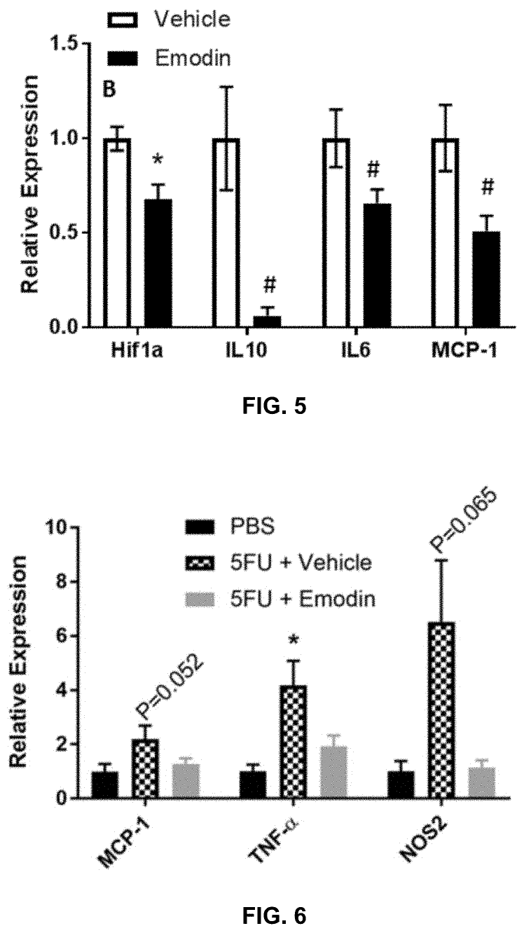

The effects of emodin on select pro-tumor markers (Hif1a, IL-10, IL-6 and MCP-1) in a tumor was also examined. Results (FIG. 5) document a decrease in relative marker expression upon treatment with emodin.

Data indicate that emodin can directly inhibit macrophage infiltration independent of tumor size that is associated with a reduction in select pro-tumor markers.

### EXAMPLE 5

The effects of dietary emodin on colon inflammation following 5FU administration were examined in a non-disease model. C57BL/6 mice were assigned to either emodin or control treatment (n=5). A phosphate buffered saline (PBS) group that did not receive either emodin or 5FU was used as a control. 5FU or PBS was administered daily for 5-days followed by a 9-day recovery (X3 cycles). The dose of 5FU was 40 mg/Kg in cycle 1 and 20 mg/Kg in cycles 2 and 3. Emodin (40 mg/Kg) was administered 3 times per week for the duration of the 5FU treatment.

Given the role of inflammation in chemotherapy-induced toxicity, an analysis of inflammatory genes from the intestines was performed. As shown in FIG. 6, mice treated with emodin show a decrease in MCP-1, TNFα, and NOS2 expression and exhibited levels similar to those of the non-5FU treated mice.

### EXAMPLE 6

Given the positive effects of emodin on inflammation associated with 5FU toxicity, use of emodin was further examined to determine any improvement in quality of life measures with emodin/5FU treatment vs. 5FU treatment alone. The effects of emodin on functional measures and survival following 5FU administration were examined in a non-disease model.

C57BL/6 mice were assigned to either emodin (40 mg/kg 3×per week; n=9) or vehicle treatment (n=10). 5FU was administered at 40 mg/kg in cycle 1 and 20 mg/kg in cycles 2 and 3. Emodin was administered 3 times per week for the duration of the 5FU treatment.

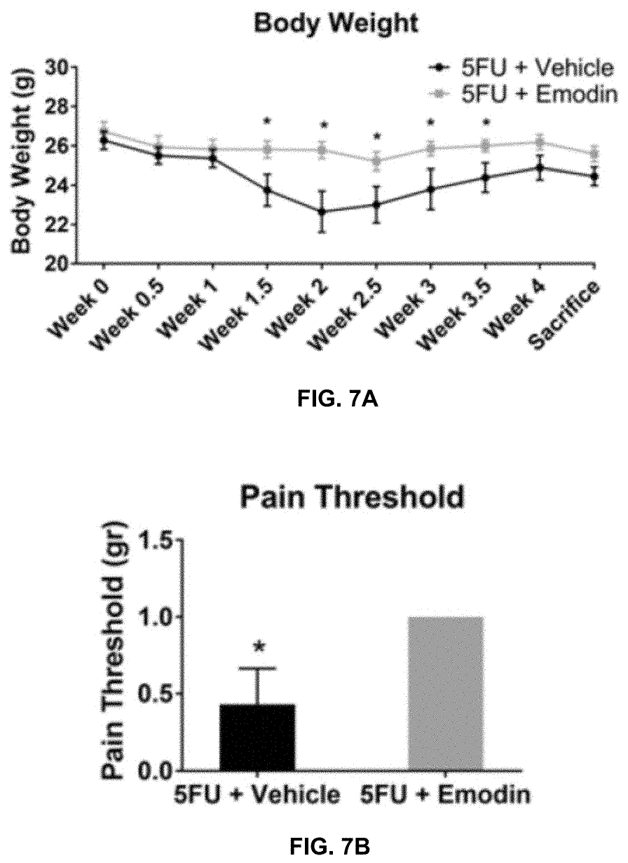

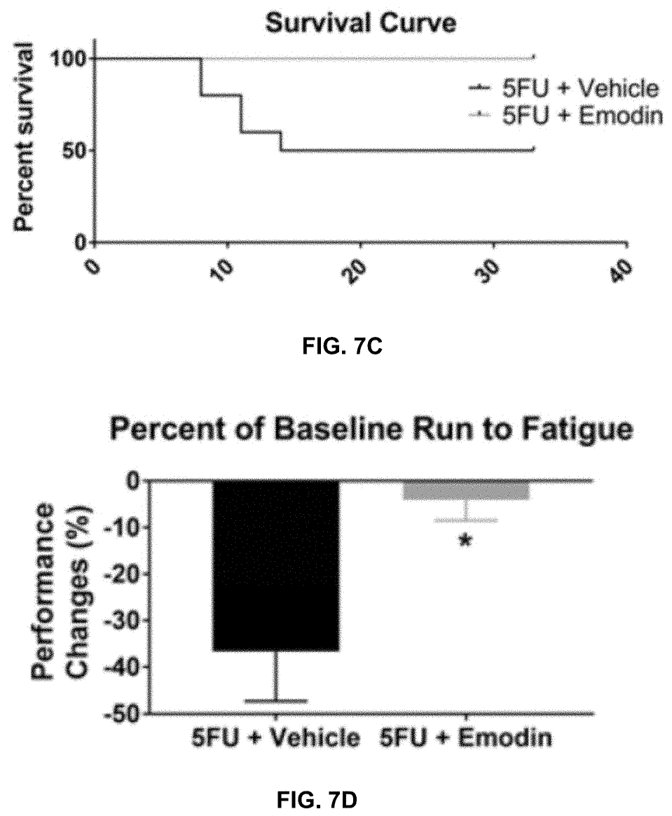

Results are shown in FIG. 7A-7D. As shown, emodin prevented the body weight loss associated with 5FU treatment (FIG. 7A) and increased survival; only 50% of mice in the vehicle group survived whereas 100% of emodin treated mice survived the 5FU regime (FIG. 7C). Following the final 5FU cycle, mice underwent a mechanical stimulation test and a run to fatigue. These data show that emodin treatment can offset the 5FU induced sensitivity to pain (FIG. 7B) and fatigability (FIG. 7D).

While certain embodiments of the disclosed subject matter have been described using specific terms, such description is for illustrative purposes only, and it is to be understood that changes and variations may be made without departing from the spirit or scope of the subject matter.

What is claimed is:

1. A method for inhibiting growth of a gastrointestinal tumor, the method comprising delivering emodin to an area comprising gastrointestinal cancer cells of the tumor.

2. The method of claim 1, wherein the emodin is delivered systemically or locally to the area.

3. The method of claim 1, wherein the gastrointestinal cancer cells comprise colorectal cancer cells, gastric cancer cells, intestinal cancer cells, or adenocarcinoma cells.

4. The method of claim 1, wherein the method is an in vivo method, and the emodin is delivered to a subject diagnosed with a gastrointestinal cancer.

5. The method of claim 4, wherein the subject is diagnosed with a colorectal cancer, an intestinal cancer, or an adenocarcinoma.

6. The method of claim 4, wherein the emodin is delivered to the subject at a dosage rate of from about 0.5 milligrams emodin per kilogram body weight of the subject per day to about 100 milligrams emodin per kilogram body weight of the subject per day.

7. The method of claim 4, wherein the emodin is delivered orally or intravenously to the subject.

8. The method of claim 1, wherein the emodin is delivered to the area in multiple doses.

9. The method of claim 1, wherein the method is an in vitro method.

10. A method for treating a gastrointestinal cancer comprising:
delivering a composition to a subject diagnosed with a gastrointestinal cancer, the composition comprising emodin; and
delivering an agent to the subject, the agent having efficacy for the gastrointestinal cancer therapy.

11. The method of claim 10, the agent comprising 5 fluorouracil, bevacizumab, irinotecan, capecitabine, cetuximab, ramucirumab, oxaliplatin, leucovorin, trifluridine and tipiracil hydrochloride, nivolumab, panitumumab, regorafenib, Ziv-Aflibercept, or any combination thereof.

12. The method of claim 10, the composition comprising the agent and the emodin.

13. The method of claim 10, wherein the composition is delivered separately from the delivery of the agent to the subject.

14. The method of claim 10, wherein the composition is delivered orally or intravenously to the subject.

15. The method of claim 10, wherein the subject is diagnosed with a colorectal cancer, an intestinal cancer, or an adenocarcinoma.

16. A method for downregulating expression level of one or more proteins involved in inflammation, the method comprising:
delivering a composition to a subject diagnosed with a gastrointestinal cancer, the composition comprising emodin; and
delivering an agent to the subject, the agent having efficacy for cancer chemotherapy, the agent upregulating expression of one or more proteins involved in inflammation; wherein
upon the delivery of the emodin, the expression of the one or more proteins is downregulated.

17. The method of claim 16, the one or more proteins comprising MCP-1, TNF-α, or NOS2.

18. The method of claim 16, the agent comprising 5 fluorouracil, bevacizumab, irinotecan, capecitabine, cetuximab, ramucirumab, oxaliplatin, leucovorin, trifluridine and tipiracil hydrochloride, nivolumab, panitumumab, regorafenib, Ziv-Aflibercept, or any combination thereof.

19. The method of claim 16, the composition comprising the agent and the emodin.

20. The method of claim 16, wherein the composition is delivered separately from the delivery of the agent to the subject.

21. The method of claim 16, wherein the subject is diagnosed with a colorectal cancer, an intestinal cancer, or an adenocarcinoma.
